(19) United States
(12) Patent Application Publication
(10) Pub. No.: US 2023/0337791 A1
(43) Pub. Date: Oct. 26, 2023

(54) A GAIT MODERATION AIDING SYSTEM

(71) Applicant: LIFESPARK TECHNOLOGIES PRIVATE LIMITED, Mumbai (IN)

(72) Inventor: Amey Devendra DESAI, Mumbai (IN)

(21) Appl. No.: 18/025,890

(22) PCT Filed: Jul. 5, 2021
(86) PCT No.: PCT/IB2021/056007

§ 371 Date: Mar. 11, 2023

(30) Foreign Application Priority Data
Jul. 13, 2020 (IN) 202021029602

(51) Int. Cl. A45B 3/00; A61B 5/11; A61B 5/00; A45B 9/02

(52) U.S. Cl. CPC A45B 3/00; A61B 5/112; A61B 5/742; A61B 5/6887; A61B 5/7455; A45B 9/02; A61B 2562/0219; A61B 2562/0247

(57) ABSTRACT

A gait moderation aiding system (100) includes a smart stick (102) and a central device (104). The smart stick (102) provides a visual cue upon detection of a gait irregularity of a user. The smart stick (102) includes an elongated housing (222), handle (201), base (204), and shaft (210). The handle (201) includes a concealed pushbutton (208) to generate vibration upon receiving a press gesture from the user. The base (204) houses a second printed circuit board (PCB) (316), a gait detection sensor (318), and a terrain detection sensor (320). The gait detection sensor (318) includes an Inertial Measurement Unit (IMU) (322) to detect the gait irregularity of the user. The shaft (210) houses a wiring harness to connect the PCBs to primary peripherals (344). The central device (104) assesses the gait irregularity of the user to provide vibratory, and auditory cueing.

### FIELD OF INVENTION

The present invention is generally related to body movement and posture support, and more particularly to a gait moderation aiding system.

### BACKGROUND OF INVENTION

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in-and-of-themselves may also be inventions.

Typically, Parkinson's disease (PD) affects the gait or the way a person walks. The changes in gait may be called Parkinson's gait or Parkinsonian gait. Gait alterations can be highly disruptive to people with PD and may interfere with the ability to work, exercise, or engage in everyday activities. Parkinson's disease patients suffer from gait abnormalities such as shuffling gait, subsequent loss of balance, and freezing of gait. In shuffling gait, steps become smaller and closer to each other. ‘Freezing’ of gait is typically observed as a rapid shortening of steps followed by the patient completely stopping and feeling unable to lift their foot. US publication US20140276242A1 filed by Chen Shyh-Min et al. discloses a wearable body sensor systems and related methods to evaluate the whole-body movement with or without an assistive walking device. Chen Shyh-Min et al. talk about monitoring the whole body movement including COG & BOS with a 3-dimensional (3D) mapping over a given period of time. The disclosed principles provide several objectives, which include providing a wearable body 3D sensor network that can constantly monitor the change of COG over the BOS during daily mobility; to clinically evaluate older adults in maintaining upright posture and prevent falls in senior living communities; and to clinically establish a quantitative mathematical model to determine the stability of the older adults. Further, US publication US2016262661A1 filed by Beccani Marco et al. discloses a clinical assessment tool coupled to a walking aid for enhancing a therapist's observation-based gait assessment with the use of additional objective and quantitative data such as acceleration, angular velocity, and applied forces. The assessment tool facilitates appropriate assistive gait device prescription, provides patients and therapists feedback during gait training, and reduces wrist and shoulder injuries with walking aid usage. The assessment tool is configured to detect the timing and speed of the walking aid, placement, angular acceleration of the walking aid, and amounts of weight borne on the walking aid.

This specification recognizes that there is a need for a gait moderation aiding system and method to provide vibratory, visual, and auditory stimuli to solve the aforementioned problems.

Thus, in view of the above, there is a long-felt need in the industry to address the aforementioned deficiencies and inadequacies.

Further limitations and disadvantages of conventional approaches will become apparent to one of skill in the art through comparison of described systems with some aspects of the present disclosure, as outlined in the remainder of the present application and regarding the drawings. In some embodiment, the numbers expressing quantities or dimensions of items, and so forth, used to describe and claim certain embodiment of the invention are to be understood as being modified in some instances by the term “about.”

### SUMMARY OF THE INVENTION

A gait moderation aiding system is provided substantially, as shown in and/or described in connection with at least one of the figures.

An aspect of the present disclosure relates to a gait moderation aiding system that includes a smart stick, a central device, and a communication application executed by a processing unit of a computing device. The smart stick provides a visual cue upon detection of a gait irregularity of a user to anticipate freezing events and preemptively start cueing to avoid/alleviate them. The smart stick includes an elongated housing, a handle, a base, and a shaft. The elongated housing includes a proximal end and a distal end. The handle is coupled to the proximal end of the elongated housing. The handle includes an ergonomic grip and a plurality of pressure sensors placed on the ergonomic grip to detect the grip and grip strength of the user. The handle includes a concealed push button to generate vibration upon receiving a press gesture from the user. In an embodiment, the vibration units are placed throughout the smart stick to generate vibration and maximize sensation in a muscle group. In an embodiment, the handle includes a first printed circuit board (PCB). The base is coupled to the distal end of the elongated housing. The base houses a second printed circuit board (PCB), a gait detection sensor, and one or more terrain detection sensors. The gait detection sensor includes a plurality of IR sensors and an Inertial Measurement Unit (IMU) to detect the gait irregularity of the user by measuring the orientation of the user's body. The terrain detection sensors include ultrasonic sensors and one or more infrared rangefinders to detect terrain to anticipate freezing events and preemptively start cueing to avoid/alleviate them. The shaft is connected with the handle to house a wiring harness to connect the first and second printed circuit boards (PCBs) to a plurality of primary peripherals. The central device is wirelessly connected with the smart stick to assess the gait irregularity of the user to provide vibratory cueing, and auditory cueing. The central device is coupled to a wearable device having a plurality of wearable form factors based on the body parts of the user. The central device provides adaptive vibratory cueing, and auditory cueing through an auditory device. The central device includes an electronic assembly to house a processor. The processor is configured to: control a plurality of secondary peripherals; execute a plurality of instructions to process sensory data received from a plurality of detectors coupled to the wearable device to assess the gait irregularity and other physiological signals of the user, wherein the detectors are housed in the electronic assembly; establish communication with the smart stick; train and run a plurality of machine learning models; actuate a plurality of transducers comprising a vibration transducer, and a sound transducer; and trigger the vibratory cueing, and the auditory cueing. The computing device is connected with the smart stick and the central device over a network. The computing device includes a memory and a processing unit configured to execute a communication application to present gait analytics to a caregiver, and a medical professional. The communication application provides a digital platform to establish an interaction between the user, caregiver, and the medical professional.

In an aspect, the base and handle of the smart stick include a microcontroller to process data captured from the sensors to output stimuli.

In an aspect, the IMU includes one or more accelerometers, one or more gyroscopes, and one or more magnetometers.

In an aspect, the base includes a flex sensor, an infrared (IR) sensor, an ultrasonic sensor, a visual sensor, and an assembly of pressure sensors to detect an angle of deflection or bending of a surface while walking.

In an aspect, the base includes a broad-spectrum camera to capture a range of wavelengths and operate in infrared (IR) waves, ultraviolet (UV) waves, and visual spectrum.

In an aspect, the smart stick includes a plurality of momentum stabilizer wheels to stabilize the smart stick upon an occurrence of freezing of gait and detecting loss of balance.

In an aspect, the base of the smart stick includes a plurality of infrared sensors, a plurality of time-of-flight radar sensors, and a plurality of ultrasonic sensors.

In an aspect, the base of the smart stick comprises a plurality of laser emitters to emit a laser for visual cueing.

In an aspect, the base includes a plurality of legs to provide a vertical support to the smart stick.

In an aspect, the base houses a battery assembly to power the PCBs, the gait detection sensor, the terrain detection sensors, vibration units of the handle, shaft, stabilizer wheels, and various sensors integrated into the smart stick.

In an aspect, the central device includes a power source to power the electronic assembly.

In an aspect, the wearable device includes a fabric.

In an aspect, the electronic assembly of the central device is arranged in between the fabric of the wearable form factors that are designed to be worn on and around one or more of the knee joint, ankle, wrist, neck, ear, and trunk.

In an aspect, the wearable form factors include one or more of a knee form factor, an ankle form factor, a wrist form factor, a neck form factor, an ear form factor, and a trunk form factor.

In an aspect, the second PCB is placed in a padded housing on the base.

In an aspect, the handle includes a plurality of mechanical buttons to adjust a distance of a laser and control at least one of an audio signal and a user interface (UI) of the computing device.

In an aspect, the handle includes a touch sensor placed on the ergonomic grip to detect a touch of the user.

In an aspect, the primary peripherals comprising a vibration unit.

In an aspect, the secondary peripherals include a sound system and a haptic vibration system.

Accordingly, one advantage of the present invention is that it utilizes patterned, and rhythmic stimuli such as vibratory, auditory, or visual to alleviate gait abnormalities/disturbances through sensory cueing.

Accordingly, one advantage of the present invention is that it provides a user-friendly and non-invasive gait moderation aiding system that can be used regularly for maximum benefit to the user.

Accordingly, one advantage of the present invention is that it is adaptive to the user's gait and makes necessary changes with disease progression.

Accordingly, one advantage of the present invention is that the digital platform provided by the communication application provides critical feedback to families helping them follow disease progression. The digital platform enables physicians, physiotherapists, and medical practitioners to virtually interact with the patients, families, and caretakers.

These features and advantages of the present disclosure may be appreciated by reviewing the following description of the present disclosure, along with the accompanying figures wherein like reference numerals refer to like parts.

### BRIEF DESCRIPTION OF THE DRAWINGS

The accompanying drawings illustrate the embodiment of systems, methods, and other aspects of the disclosure. Any person with ordinary skills in the art will appreciate that the illustrated element boundaries (e.g., boxes, groups of boxes, or other shapes) in the figures represent an example of the boundaries. In some examples, one element may be designed as multiple elements, or multiple elements may be designed as one element. In some examples, an element shown as an internal component of one element may be implemented as an external component in another and vice versa. Furthermore, the elements may not be drawn to scale.

Various embodiment will hereinafter be described in accordance with the appended drawings, which are provided to illustrate, not limit, the scope, wherein similar designations denote similar elements, and in which:

FIG. 1 illustrates a network implementation of a gait moderation aiding system, in accordance with at least one embodiment.

FIG. 2 illustrates a perspective view of the smart stick, in accordance with at least one embodiment.

FIG. 3 illustrates a block diagram of the communication between the computing device and various components of the smart stick and the central device, in accordance with at least one embodiment.

FIG. 4 illustrates a block diagram of a software architecture used by the present gait moderation aiding system, in accordance with at least one embodiment.

FIG. 5 illustrates a perspective view of cutaneous patterns of the wearable form factors, in accordance with at least one embodiment.

FIG. 6 illustrates a perspective view of electronic assembly, in accordance with at least one embodiment.

FIG. 7 illustrates a perspective view of a knee support structure of a knee form factor having a fabric base shape of wrap-around type, in accordance with at least one embodiment.

FIG. 8 illustrates a perspective view of an electronic housing section of the knee form factor having the fabric base shape of wrap-around type, in accordance with at least one embodiment.

FIG. 9 illustrates a perspective view of a supporting mesh structure of the knee form factor having the fabric base shape of wrap-around type, in accordance with at least one embodiment.

FIG. 10 illustrates a perspective view of a knee support structure of a knee form factor having a fabric base shape of sock type, in accordance with at least one embodiment.

FIG. 11 illustrates a perspective view of an electronic housing section of the knee form factor having the fabric base shape of sock type, in accordance with at least one embodiment.

FIG. 12 illustrates a perspective view of a supporting mesh structure of the knee form factor having the fabric base shape of sock type, in accordance with at least one embodiment.

FIG. 13 illustrates a perspective view of a band form factor, in accordance with at least one embodiment.

FIG. 14 illustrates a perspective view of a neck form factor, in accordance with at least one embodiment.

FIG. 15 illustrates a perspective view of an auditory device, in accordance with at least one embodiment.

FIG. 16 illustrates a perspective view of a laser and servo assembly, in accordance with at least one embodiment.

### DETAILED DESCRIPTION OF THE EMBODIMENTS HEREIN

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Methods of the present invention may be implemented by performing or completing manually, automatically, or a combination thereof, selected steps or tasks. The term “method” refers to manners, means, techniques, and procedures for accomplishing a given task including, but not limited to, those manners, means, techniques, and procedures either known to or readily developed from known manners, means, techniques, and procedures by practitioners of the art to which the invention belongs. The descriptions, examples, methods, and materials presented in the claims and the specification are not to be construed as limiting but rather as illustrative only. Those skilled in the art will envision many other possible variations within the scope of the technology described herein.

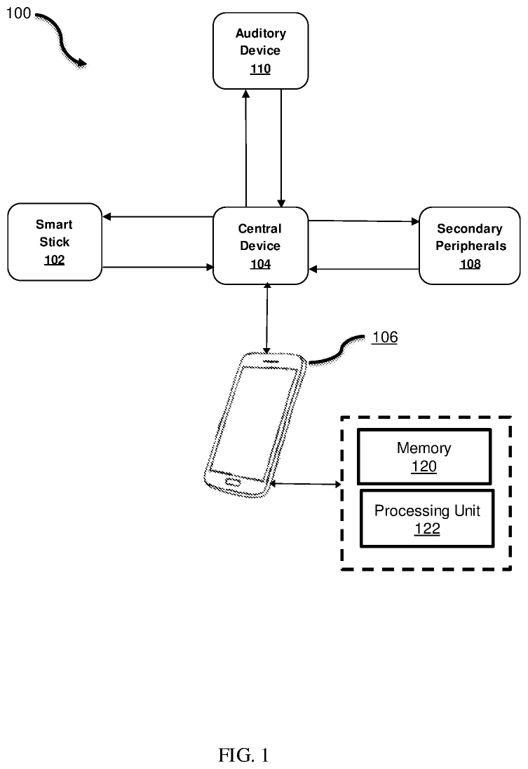

FIG. 1 illustrates a network implementation of a gait moderation aiding system 100, in accordance with at least one embodiment. The gait moderation aiding system 100 includes a smart stick 102, a central device 104, and a communication application executed by a processing unit 122 of a computing device 106. The smart stick 102 provides a visual cue upon detection of a gait irregularity of a user. The central device 104 is wirelessly connected with the smart stick 102 to assess the gait irregularity of the user to provide a vibratory cueing, and an auditory cueing. The smart stick 102 transmits the sensor data, logging data, and emergency (SOS) reporting data to the central device 104. The central device 104 transmits SOS control data, timestamp synchronization data, and parameter synchronization data to the smart stick 102. Further, the central device 104 control the smart stick 102 and provides haptic control, laser control, and sound control to the smart stick 102. The central device 104 provides adaptive vibratory cueing, and auditory cueing through an auditory device 110. Further, the central device 104 transmits the audio data and triggers the signal to the auditory device 110. The auditory device 110 transmits the sensor data and status indicators to the central device 104.

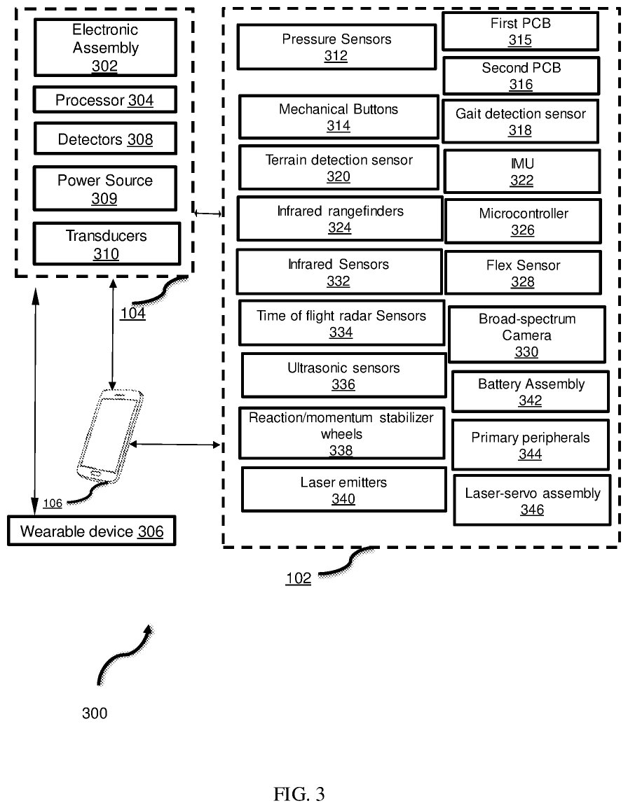

The central device 104 includes an electronic assembly 302 (shown in FIG. 3) to house a processor 304 (shown in FIG. 3). The processor 304 is configured to control a plurality of secondary peripherals 108. The central device 104 provides haptic control, timestamp synchronization data, and parameter synchronization data. The secondary peripherals 108 provides sensor data, and logging data to the central device 104. The computing device 106 includes a memory 120 and a processing unit 122 configured to execute a communication application to present gait analytics to a caregiver, and a medical professional. The communication application provides a digital platform to establish an interaction between the user, caregiver, and the medical professional. In an embodiment, the communication application is a mobile application that is based on various operating systems such as Android®, Windows®, and iOS®. Examples of the computing device 106 including but are not limited to, a portable computer, a personal digital assistant, a handheld device, and a workstation. The mobile application serves as an interface to a software platform that provides gait analytics to medical professionals and caretakers/family members. In an embodiment, the mobile application provides a platform to communicate with healthcare professionals through chat or voice/video calls. Also, the mobile application provides a method to order medication online. The mobile application provides a scheduler to book appointments with healthcare professionals. Finally, the mobile application also provides informative media about topics such as lifestyle, disease progression, new therapies, etc. The mobile application performs the analysis either on computing device 106 or offloaded to a cloud device. In an embodiment, the mobile application provides an avenue for engaging activities such as multiplayer games, webinars, mental health activities, etc.

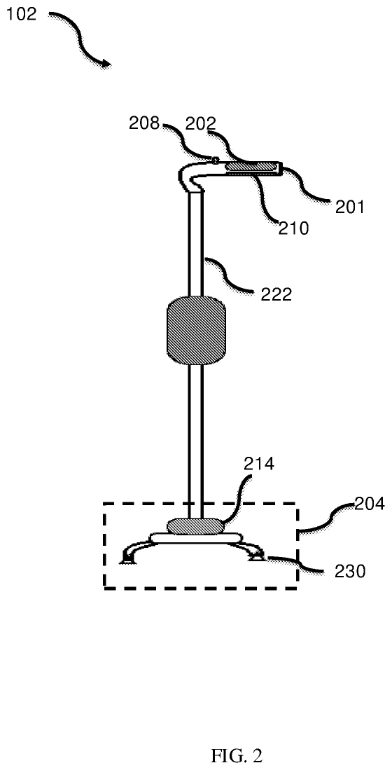

FIG. 2 illustrates a perspective view of the smart stick 102, in accordance with at least one embodiment. The smart stick 102 includes an elongated housing 222, a handle 201, a base 204, and a shaft 210. The elongated housing 222 includes a proximal end and a distal end. The handle 201 is coupled to the proximal end of the elongated housing 222. The handle 201 includes an ergonomic grip 202 and a plurality of pressure sensors 312 (shown in FIG. 3) placed on the ergonomic grip 202 to detect the grip and grip strength of the user. The handle 201 includes a concealed push button 208 (SOS button) to generate vibration upon receiving a press gesture from the user. In an embodiment, the handle 201 includes a plurality of mechanical buttons 314 (shown in FIG. 3) to adjust a distance of a laser and control at least one of an audio signal and a user interface (UI) of the computing device 106. In an embodiment, the handle 201 includes a touch sensor placed on the ergonomic grip 202 to detect a touch of the user. In an embodiment, the handle 201 includes sensors to detect a plurality of other physiological functions such as pulse/heartrate, temperature, glucose levels, and blood pressure. In an embodiment, the handle 201 includes a first printed circuit board (PCB) 315.

The base 204 is coupled to the distal end of the elongated housing 222. The base 204 includes a housing 214 to house a second printed circuit board (PCB) 316, a gait detection sensor 318, and one or more terrain detection sensors 320. The gait detection sensor 318 includes an Inertial Measurement Unit (IMU) 322 to detect the gait irregularity of the user by measuring the orientation of the user's body. In an embodiment, the IMU 322 includes one or more accelerometers, one or more gyroscopes, and one or more magnetometers. The terrain detection sensors 320 include one or more infrared rangefinders 324 to detect terrain to provide the visual cue to guide a user while walking. In an embodiment, the base 204 of the smart stick 102 includes a microcontroller 326 to process data captured from the sensors to output stimuli. In an embodiment, base 204 includes a flex sensor 328 to detect an angle of deflection or bending of a surface while walking. In an embodiment, the base 204 includes a broad-spectrum camera 330 to capture a range of wavelengths and operate in infrared (IR) waves, ultraviolet (UV) waves, and visual spectrum.

In an embodiment, the base 204 of the smart stick 102 includes a plurality of infrared sensors 332, a plurality of time-of-flight radar sensors 334, and a plurality of ultrasonic sensors 336. In an embodiment, the smart stick 102 includes a plurality of momentum stabilizer wheels 338 to stabilize the smart stick 102 upon an occurrence of freezing of gait and detecting loss of balance.

In an embodiment, the base 204 of the smart stick 102 includes a plurality of laser emitters 340 to emit a laser for visual cueing. In an embodiment, base 204 includes a plurality of legs 230 to provide a vertical support to the smart stick 102. In an embodiment, the base 204 houses a battery assembly 342 to power the first and second PCBs 315 and 316, the gait detection sensor 318, and the terrain detection sensors 320. In an embodiment, the second PCB 316 is placed in a padded housing on base 204.

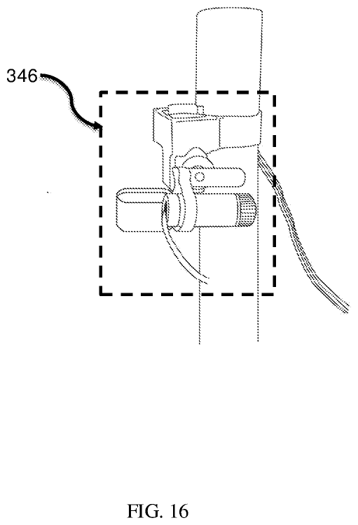

The shaft 210 is connected with the handle 201 to house a wiring harness to connect the first and second printed circuit boards (PCBs) 315 and 316 to a plurality of primary peripherals 344. In an embodiment, the primary peripherals 344 comprising a vibration unit. In an embodiment, the terrain detection sensors 320 are mounted throughout the smart stick 102. In an embodiment, the elongated housing 222 acts as a main shaft and base that includes a laser-servo assembly 346 (shown in FIG. 16) which is mounted on the elongated housing 222. In an embodiment, the shaft 210 is coupled at a bottom portion of the handle 201. In an embodiment, the laser-servo assembly 346 includes a laser and a servo motor. The laser is configured to emit light as a line on the floor and the line is used for purpose of visual cueing. In an embodiment, the servo is configured to control the position of the laser. Thus, the servo is configured to control the position and orientation of the line with respect to the stick.

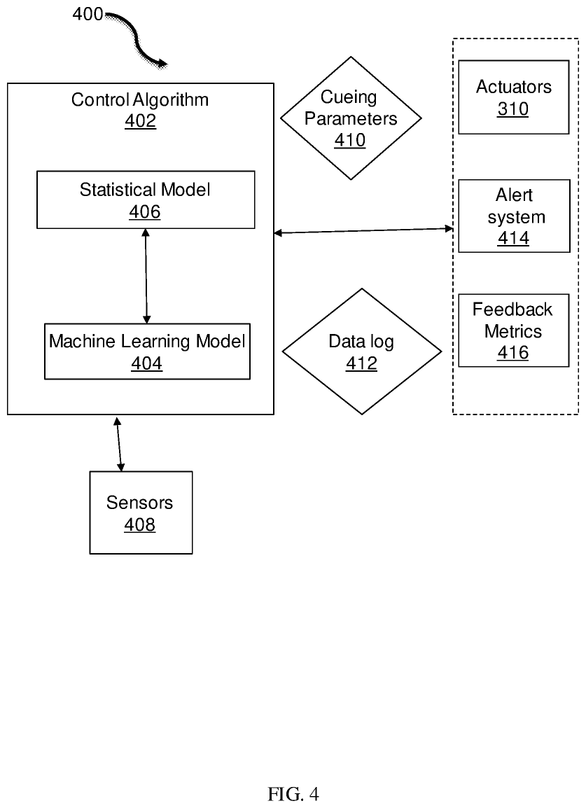

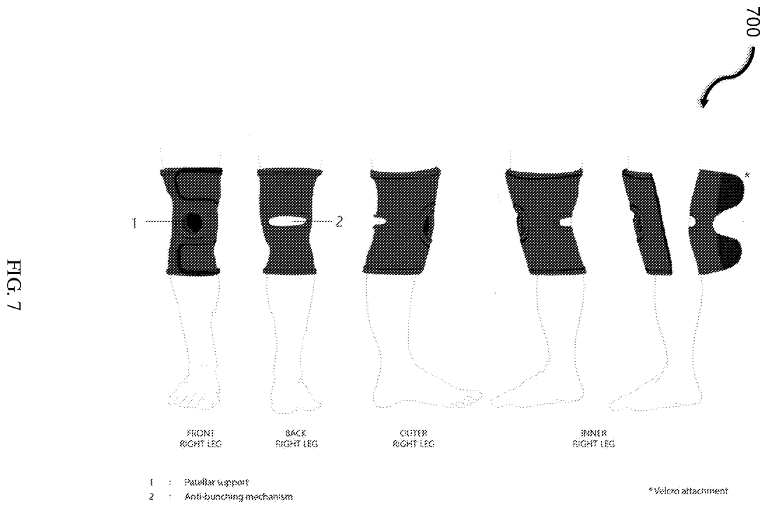

FIG. 3 illustrates a block diagram of the communication between the computing device 106 and various components of the smart stick 102 and the central device 104, in accordance with at least one embodiment. The central device 104 is coupled to a wearable device 306 having a plurality of wearable form factors (shown in FIG. 7-FIG. 15) based on the body parts of the user. The central device 104 includes an electronic assembly 302 to house a processor 304. The processor 304 is configured to: control the secondary peripherals 108; execute a plurality of instructions to process sensory data received from a plurality of detectors that includes various sensors attached with the central device 104 coupled to the wearable device 306 to assess the gait irregularity of the user, wherein the detectors are housed in the electronic assembly 302; establish a communication with the smart stick 102; train a plurality of machine learning models 404 (shown in FIG. 4); actuate a plurality of transducers 310 (actuator) comprising a vibration transducer, and a sound transducer; and trigger the vibratory cueing, and the auditory cueing. In an embodiment, the computing device 106 may connect with the smart stick 102 and the central device 104 over a network. Network may be a wired or a wireless network, and the examples may include but are not limited to the internet, Wireless Local Area Network (WLAN), Wi-Fi, Long Term Evolution (LTE), Worldwide Interoperability for Microwave Access (WiMAX), and General Packet Radio Service (GPRS). In an embodiment, the secondary peripherals 108 include a sound system and a haptic vibration system.

In an embodiment, the central device 104 includes a power source 309 to power the electronic assembly 302. Examples of the power source 309 including but are not limited to an external power source or a battery to supply power to the various components of the central device 104.

In an embodiment, the wearable device 306 includes a fabric. In an embodiment, the electronic assembly 302 of the central device 104 is arranged over the wearable device 306 that is designed to be worn on and around one or more of the knee joint, ankle, wrist, neck, ear, and trunk. In an embodiment, the wearable form factors include one or more of a knee form factor, an ankle form factor, a wrist form factor, a neck form factor, an ear form factor, and a trunk form factor.

The wearable device 306 provides adaptive vibratory cueing, provides auditory cueing through earphones or auditory devices wirelessly or in a wired manner, and logs gait data to build a balance profile for the user. The wearable device 306 uses gait data to adapt to the user's gait and dynamically adjust stimulus patterns to benefit the user the most. In an embodiment, the smart stick 102 includes a strap to secure the elastic grip to the user's hand. The smart stick 102 estimates the risk of falling due to surrounding terrain and communicates with the wearable device 306 to alter stimulus patterns to compensate. In an embodiment, the smart stick 102 identifies factors in the external environment of the patient that may cause the patient to fall. The factors include but not limited to doorways or narrow paths, change in the floor surface, e.g., from carpet to tile, and obstacles in the path.

The terrain detection method in the smart stick 102 works to detect such instances that cause FOG and prevent falls. In an embodiment, the smart stick uses a distance measurement sensor, a Surface detection sensor, or a combination thereof. Examples of distance measurement sensors include but are not limited to infrared sensors, ultrasonic sensors, lidar sensors, laser ToF sensors, etc. Examples of surface detection sensors include but are not limited to force/pressure sensors, capacitive touch sensors, mechanical switches, etc. Further, the smart stick uses cameras operating in visual, IR, ultraviolet (UV), or any other spectrum. Through these sensors, the smart stick detects if there is an obstacle along the way, or if there are other factors that might cause falls. Sensors are arranged to cover 180 degrees (field-of-view) in the front of the user.

In operation, if the sensors of the smart stick 102 detect obstacle or narrowing or surface change, the sensors estimate the distance from the obstacle and check for unsteadiness in gait. If unsteady beyond a threshold, the smart stick starts/increases the intensity of the auditory and visual stimulus and alerts the helper or caregiver. Then the smart stick 102 sends a notification on the phone/other computing device and the phone/other computing device may generate a notification signal such as a beep, wherein the notification enables the other device such as knee module, if present in the vicinity, to start cueing with the smart stick 102 in synchronization. Lastly, the smart stick 102 adapts stimulus according to the gait pattern.

In an embodiment, the smart stick 102 predicts the occurrence of freezing of gait (FOG). Typically, FOG is not a binary phenomenon, but rather has gradations in practice. Some obvious markers are shortening of stride length, faster stepping, and unsteadiness in gait. The smart stick tracks gait, and along with the data from the wearable device 306, checks for such markers to predict the onset of FOG. Also, the terrain detection algorithm identifies areas that may potentially cause freezing, and the stick uses this information to take suitable preventive measures

In operation, the wearable device 306 sends vibratory stimuli and auditory stimuli. The smart stick actuates visual and auditory stimuli. Further, the smart stick 102 provides vibratory cueing through the grip and includes a strap to secure the grip to the user's hand. Each can sense the user's gait independently using onboard sensors. The smart stick 102 can stimulate the user using visual and auditory cues with or without the presence of the wearable device 306 and vice versa. When both are present simultaneously, they communicate with each other to supplement their functions. For example, with only the smart stick, when a potential freezing scenario is detected, the visual and auditory cues are presented in a specific pattern. If the wearable device 306 is also present, the vibratory pattern is also altered.

In an embodiment, the balance profile is an analysis of the data collected from the user using both the smart stick and the wearable device 306. Further, the smart stick includes sensors to detect grip strength on the handle, an SOS self-reporting button, and IMUs as well as other terrain mapping sensors to detect loss of balance. Similarly, the wearable device 306 has sensors to detect loss of balance independently. When both are present simultaneously, they combine their predictions to make an accurate diagnosis of balance.

FIG. 4 illustrates a block diagram of a software architecture 400 used by the present gait moderation aiding system 100, in accordance with at least one embodiment. According to an embodiment herein, the gait moderation aiding system 100 utilizes a control algorithm 302 that includes a machine learning model 404 and statistical model 406 or statistical algorithms to carry out the data analysis on the data received from the various sensors 408 of the present gait moderation aiding system 100. Examples of the statistical methods include but not limited to trend calculation in gait length, and speeds; and trend calculation in gait asymmetry. Examples of machine learning include but are not limited to unsupervised clustering of gait patterns, and supervised prediction of fall risk based on sensor data. Trained models are then sent back to the communication application which updates them in the processor, processing unit, and microcontrollers. These models are then applied during daily use to adjust cueing parameters 410 based on the data log 412. According to an embodiment herein, the control algorithm 302 further controls the actuators 310, an alert system 414 such as the vibration unit, audio unit, and display unit, and also provides feedback metrics 416.

In an embodiment, various open-source programming languages and libraries such as Python, C, C++, Java, TensorFlow, SQL and variants, JavaScript, and variants are used to build the digital/software platform. Each processor, processing unit, and microcontroller includes a machine learning model. The data reported by the sensors 408 are used by these models to predict a few quantities such as gait frequency, and fall risk score. Gait frequency is used to adjust the frequency of cueing, to make it easier for the user to synchronize. The fall risk score is based on sensor output as well as historical trends. Typically, a dose of medicine is effective for a certain period, after which symptoms return. Thus, there are times during the day when users are more likely to have problems, which the model takes into account when predicting fall risk. In an embodiment, the processor, processing unit, and microcontroller utilizes a global system for mobile communication (GSM) module or a General Packet Radio Service (GPRS), or a Wi-Fi module or a Bluetooth® module for transmission of data.

In an embodiment, the wearable device 306 having various wearable form factors or modules based on the body parts such as the knee, ankle trunk, wrist, neck, and ear of the user. The wearable form factors are used to apply sensory cueing. In an embodiment, the modules are categorized as band module, knee module, ankle module, trunk module, wrist module, neck module, and sound module. Sensory cueing is applied by rhythmic vibration, sound, or light. Each of these wearable form factors or modules can function independently. When more than one is operational, they communicate with each other to synchronize cueing. When a module includes a pair of devices, such as knee, ankle, wrist, or sound modules, each of the units can function independently or in pair mode by communicating with each other to synchronize cueing. These modules may communicate with a phone on occasion to relay sensory data. They contain batteries as a power source.

In an embodiment, the band module, knee module, ankle module, trunk module, wrist module, neck module, and sound module perform various functions such as collecting and storing IMU data, communicating with any other devices in the surrounding, communicating with mobile phones, administering cueing, and execute control and machine learning algorithms to assess gait, status and cueing.

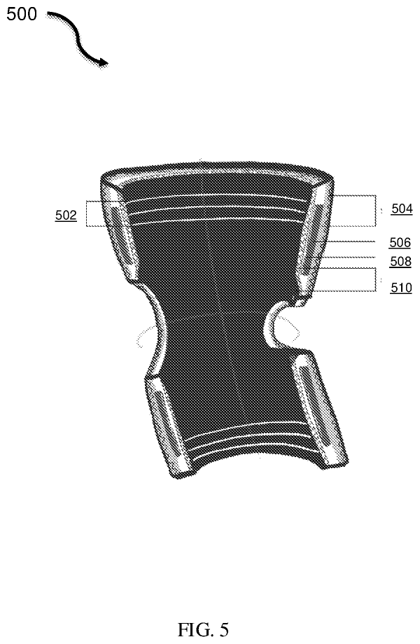

Each module adapts to the individual user, modifying its cueing pattern as per the user. The main electronics in each module consist of the central module (described below), vibration or sound transducers, battery apparatus, and its fabric or other structure. Each module is described in detail in conjunction with FIG. 5 to FIG. 15.

In an embodiment, the wearable device 306 is used for the application of sensory cueing. The wearable device 306 applies to cues through three physical mediums: vibration, sound, and light.

In an embodiment, the central device 104 is the primary wearable. The central device 104 consists of an electronic assembly 302 or an electromechanical assembly that controls peripherals such as the sound system and haptic vibration system. The central device 104 also runs the control algorithms which manage functions such as sensor I/O, wired/wireless communications, machine learning models, and actuation of transducers. While the core electronic components are the same, their physical arrangement takes multiple forms.

The electronic assembly 302 in the central module consists of microprocessors, communications hardware capable of Bluetooth/BLE/Wi-Fi/GSM/4G/radio/various near field technologies/etc, haptic vibration unit controllers (of different forms), audio drivers/controllers, voltage converters, memory controllers, memory interfaces, RAM modules, Inertial Measurement Units (IMUs), electrical connectors, wire-to-board connectors/wire-to-wire connectors, speakers, optical sensors, pressure sensors, stretch sensors, flex sensors, current and voltage sensors, temperature sensors, moisture sensors, conductivity/resistivity sensors, electromyographic sensors, electromyographic electrodes, electronic amplifiers, signal processing modules, buttons, batteries and charging apparatus, etc. The electromechanical assembly consists of haptic vibration transducers, sound transducers, heating/cooling modules, display screens, glucose monitors, heart-rate monitors, blood pressure monitors, apparatus to measure vital physiological signs, etc.,

The present disclosure describes the structure of the knee sleeve or the knee form factor. In the knee form factor, the sensors and components of the central module are arranged over the wearable device that is designed to be worn on and around the knee joint. Electronics are embedded within a fabric structure to create a wearable form. A blend of natural and synthetic materials is used to make the fabric structure. The fabric base is stretchable and flexible. The fabric is knitted with a large weave, to make it extremely breathable. Above the fabric layer, there are heat conductive fibers that are used to create warmth in different parts of the brace. The fibers are spread throughout the brace, in a mesh network. Heat is conducted from the central modules' heating element to the entire network.

In an embodiment, the fabric base is divided into three sections, the knee support section, the electronic housing section, and the supporting mesh. The knee support section has slightly more tension as compared to the other sections. This section is centered around the knee and has several support structures to apply even pressure on the knee joint and provide appropriate support while walking. The central part of the knee support section has a snap lock and zip combination to adjust the tension in the support structure.

The electronic housing section is located around the vibration units, the battery housing, central module, and flex-PCB/wire harness routing areas. This section has a tighter weave and is more rigid than other sections. The electronics are mounted on this section. It has less tension than the knee support section.

The supporting mesh fills the spaces between the other sections and completes the traditional knee brace cylindrical shape. This section is made of a large weave, low-density mesh. It looks similar to netting and has slightly lower tension than the electronic housing section.

A detailed drawing of the form of the fabric structure and component arrangement is shown below in FIGS. 7 to 12. FIG. 5 illustrates a perspective view 500 of cutaneous patterns 502 of the wearable form factors, in accordance with at least one embodiment. The inner surface of the fabric, which directly touches the user's skin, has a pattern of lines and dots embroidered on it. The patterns are embossed on the fabric surface and press into the skin. This pattern improves the sensation of skin stretch and movement of/around the joint in general. Additionally, the pattern improves the proprioception of the joint. The pattern is situated throughout the knee brace. The cutaneous patterns 502 of the wearable form factor (knee module) are placed 1-2 centimeters apart. The cutaneous patterns 502 includes a supporting mesh structure 504, a vibration unit 506, a heat-conducting fibre 508, and a knee support structure 510.

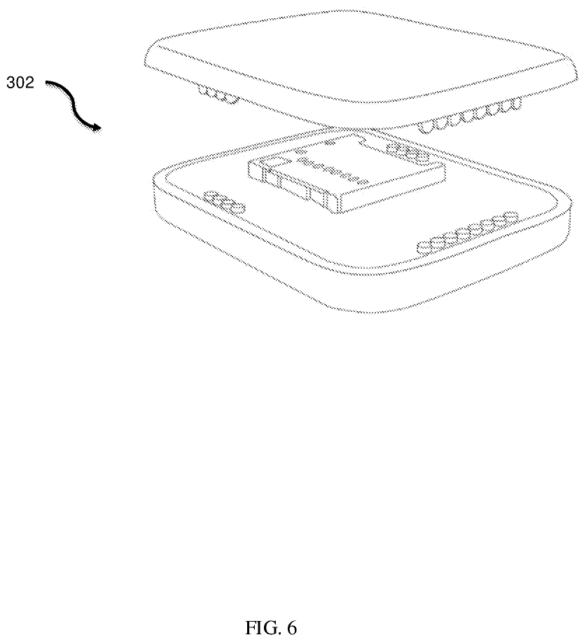

FIG. 6 illustrates a perspective view of electronic assembly 302, in accordance with at least one embodiment. The front of the wearable device 306 such as the knee brace is covered with impact-absorbing material. This material is layered over the fabric base and electronic assembly. In an embodiment, the present disclosure describes the arrangements of the components in the electronic assembly 302. The main PCB assembly contains either a standard rigid or rigid-flex PCB attached to a custom wire-to-board connector. The connector is at the end of either a flex PCB substrate or silicone wire harness. The flex PCB/wire harness is connected to the vibration units and the battery pack. The connector is integrated inside a shell that covers the rigid part of the PCB. The external part of the shell has a pressure-sensitive, touch-sensitive surface that can be used to control power, wireless connectivity, and other functions. This surface may also have buttons and indicator lights. The shell has magnets arranged in a pattern throughout its body to facilitate alignment with an external dock. This external dock may be used for charging, data transfer, communications, etc.

The vibration units are arranged in a specific pattern around the mid-thigh and the upper calf, to maximally vibrate the surrounding muscles and facilitates long-term nervous system adaptation. The present gait moderation aiding system target the quadriceps muscles, hamstring muscles, tibialis, gastrocnemius, and soleus muscles as well as cutaneous receptors. Besides the main PCB assembly, there is a heating element isolated from the main assembly and shell by a heat-insulating material. The heating element will disperse heat upon activation by the central control unit via the heat-conducting fibers.

The battery pack may be composed of lithium-ion, lithium polymer, lithium ceramic, or other battery technology. It is connected to the main PCB assembly through a wire harness. A display may be located on the outer surface of the shell. The display has pressure-sensitive, touch-sensitive technology to enable interaction.

The entire electronic assembly 302 is covered by a thin fabric layer, with gaps to expose the charging and maintenance areas on the main shell, battery pack, and motors.

FIG. 7 illustrates a perspective view of a knee support structure 700 of a knee form factor having a fabric base shape of wrap-around type, in accordance with at least one embodiment. The wrap-around type of the knee form factor has an adjustable, push-to-lock mechanism. The mechanism is embedded in the fabric base. The lock snaps to fit into the receptacle. There are guide magnets that help with the alignment of the receptacle and locking pin. The direction of looking is normal (perpendicular) to the leg. The locking mechanism is placed on the knee support section of the brace. FIG. 7 depicts various views of the legs using the knee support structure 700 that includes a patellar support 1, an anti-bunching mechanism 2, and a Velcro® attachment 702.

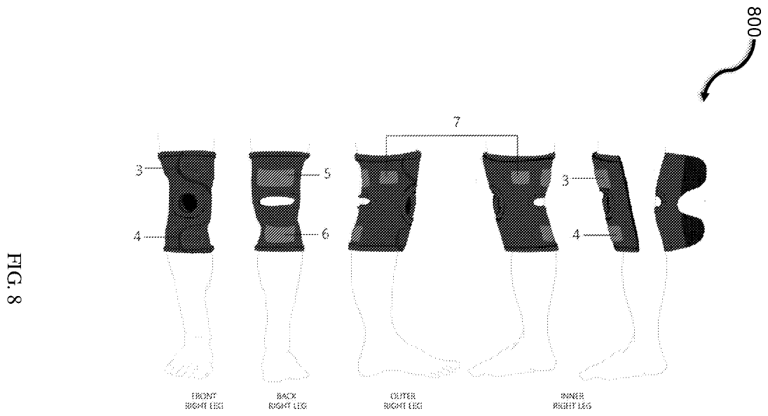

FIG. 8 illustrates a perspective view 800 of an electronic housing section of the knee form factor having the fabric base shape of wrap-around type, in accordance with at least one embodiment. The electronic housing section 800 of the knee form factor depicts the positions of battery housing unit 3, various vibration units (4,5,6), and a central control unit/power unit 7. In an embodiment, all the units linked at the bottom with heat conductive fibers transfer heat through the cloth.

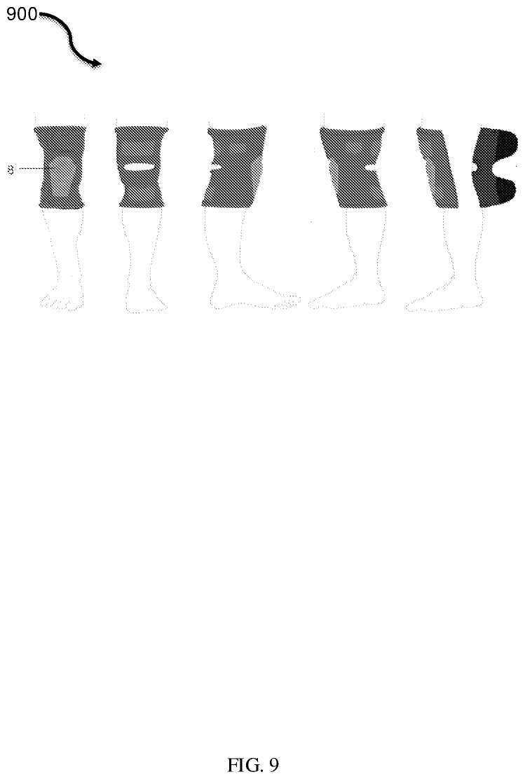

FIG. 9 illustrates a perspective view 900 of a supporting mesh structure of the knee form factor having the fabric base shape of wrap-around type, in accordance with at least one embodiment. The supporting mesh structure of the knee form factor depicts the position of impact-absorbing material.

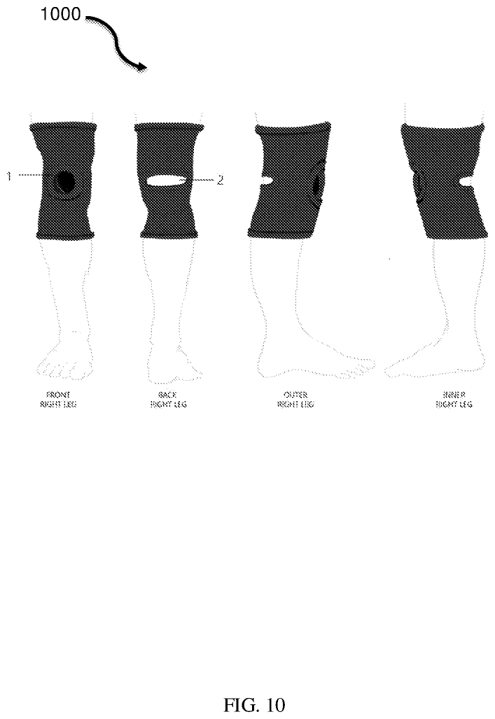

FIG. 10 illustrates a perspective view of a knee support structure 100 of a knee form factor having a fabric base shape of sock type, in accordance with at least one embodiment. FIG. 10 depicts positions of a patellar support 1, and an anti-bunching mechanism 2 in the knee support structure 1000 in conjunction with various views of the legs.

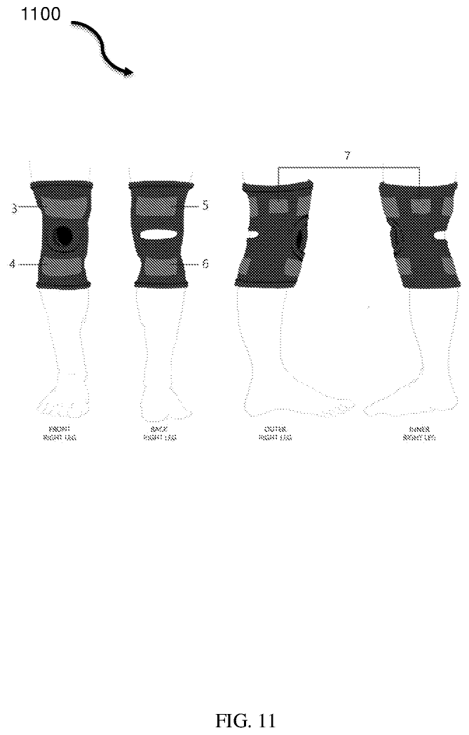

FIG. 11 illustrates a perspective view 1100 of an electronic housing section of the knee form factor having the fabric base shape of sock type, in accordance with at least one embodiment. The electronic housing section of the knee form factor depicts the positions of battery housing unit 3, various vibration units (4,5,6), and a central control unit/power unit 7. In an embodiment, all the units linked at the bottom with heat conductive fibers transfer heat through the cloth.

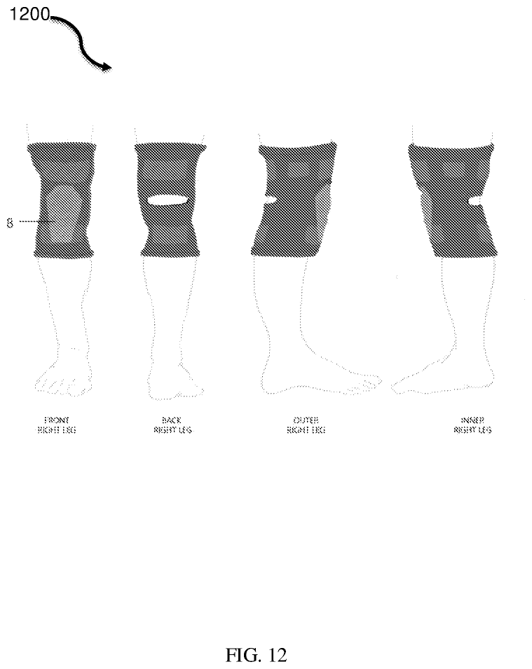

FIG. 12 illustrates a perspective view 1200 of a supporting mesh structure of the knee form factor having the fabric base shape of sock type, in accordance with at least one embodiment. The supporting mesh structure of the knee form factor depicts the position of impact-absorbing material.

In an embodiment, the knee form factor or the knee module carries out various functions such as gait data collection, communication with other modules, real-time gait data processing, executing machine learning algorithms, performing sensory cueing e.g. vibration, sound, and temperature regulation. The knee module can act as either the central controller or the peripheral device depending on initialization. When there is only one knee module in use, it defaults to acting as the central device. When it is the central device, it carries out all the basic functions described above. In addition, if a smart stick is in use, it assumes master control over the stick. When two knee modules are in operation, one behaves as the central controller and the other as the peripherals. Both carry out the same basic functions listed above. The primary difference is in the calculation and control of cueing. The peripherals gather data and send relevant information to the central module. The central device combines this information with its own collected data, preprocesses this information, feeds it through different algorithms including the machine learning algorithms, and prepares the cueing pattern. It controls haptic cues in its module and sends a signal to the peripheral module to turn its haptics on/off. The central device may also directly control the sound modules, or the control signal to the sound modules may be relayed by the peripheral smart/smart stick or stick module. Both modules individually run algorithms to detect critical events such as gait/postural instability falls, gait initiation, etc. Either module can declare an “SOS”, in which event an emergency cueing pattern is initiated which is controlled by the central module.

In an embodiment, the smart stick and all the modules may also be used to manually trigger an “SOS”. In the presence of the stick module, the central module collects terrain information, gait information, grip strength, gait/postural stability data, etc. from the stick. It also controls visual cueing through the stick.

There are various modes of operation of the above-described modules such as i) single-mode, ii) single and smart stick, iii) single and sound module, iv) single, smart stick and sound module, v) dual mode, vi) dual and smart stick, vii) dual and sound mode, viii) dual, smart stick, and sound module.

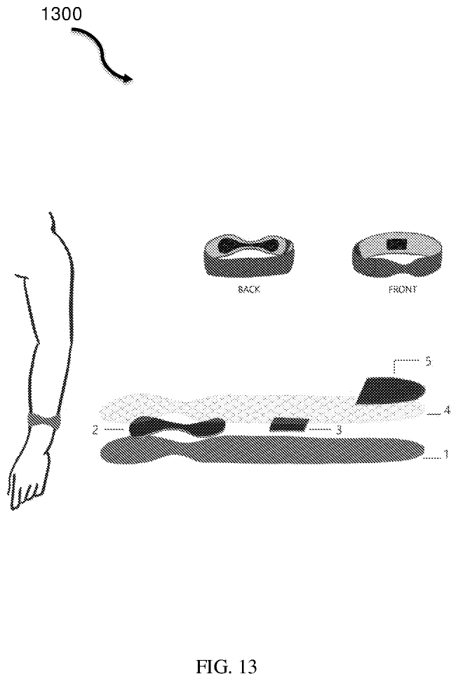

FIG. 13 illustrates a perspective view 1300 of a band form factor, in accordance with at least one embodiment. In an embodiment, the band form factor is a single strap-like wearable worn over the thigh, shin, ankle, arm, trunk, or neck. It consists of the central device, vibration units 2, and other electronics embedded within a supporting structure. It is elastic and has hook and loop type fasteners combined with a locking mechanism. Vibration targets quads and hams. The band form factor has all the sensors which are part of the central device. The band form factor includes an outer mesh 1, a battery housing unit, vibration unit, and central control unit/power unit 3, inner mesh 4, and hook and loop fastener 5 such as Velcro® attachment.

According to an embodiment herein, in the ankle form factor, the components are arranged over the wearable device that is designed to be worn on and around the ankle joint. Electronics are embedded within a fabric structure to create a wearable form. There are two versions of the ankle module: sock-type and wrap-around.

In an embodiment, the ankle module is shaped like the sock type. It is composed of three layers namely i) the fabric base, ii) the electronic assembly layer, iii) cushioning layer.

The fabric base is similar in structural design to the knee module. It consists of a closer-knit, higher tension support area, a large weave & medium tension electronic assembly section, and a supporting structure with a large weave and comparatively lower tension. The arrangement of different areas is illustrated below for both types of ankle modules.

The electronic assembly layer houses the main PCB shell, battery pack, and vibration units. The electronics are described in detail in the following section. The electronic assembly layer is located above the corresponding electronic assembly section on the fabric base. It holds the various components of the electronic assembly in place and provides support and protection for them.

The cushioning layer provides supporting structures for areas on and around the ankle joint. Specifically, it provides cushioning for the heel, cushioning and support for the arch and general support for the sole. The arch support is composed of a conformable material that adapts to the arch shape. The heel support is a soft, conformable material that continues from the arch support and goes up to the lower ankle joint to support the heel completely.

The wrap-around ankle module features a semi-rigid outer structure that provides a foot-like shape to the ankle module when not worn. It maintains this structure in the absence of any interaction with other objects, making it possible for the module to stand upright and facilitate the user in stepping into the module with ease. Upon stepping in, the module can be attached to the foot using the locking mechanism.

The electronic assembly of the wrap-around ankle module is identical to the knee module except in one aspect. The number of vibration units is different, there are at most two vibration units. The vibration units are arranged to maximize sensation in major muscle groups around the ankle joint. We target the tibialis anterior, gastrocnemius, soleus, digitorium, hallucius, peroneus, and other muscles in the area as well as cutaneous receptors.

For the wrap-around variant of the ankle module, a wide hook and loop type of arrangement facilitates locking. In addition, there may be a buckle arrangement similar to the straps on a wristwatch, but with a wider locking pin. The locking mechanism is present around the joint to minimize interaction with the joint during movement. An illustration is attached below.

According to an embodiment herein, in the wrist form factor, the components are arranged over a wearable that is designed to be worn on the lower arm around the wrist. Electronics are embedded within a fabric-plastic hybrid structure to create a wearable form.

The wrist module is shaped like a circular band. It is composed of non-elastic segments and elastic segments. The elastic segments allow it to stretch and grip on the user's wrist and arm. The wrist module has two main sections such as fabric hybrid base, and electronic assembly.

The fabric hybrid base is divided into a set of segments. Each segment is lined with a breathable fabric on the inner side of the band which touches the skin. Segments are connected by an elastic material.

The electronic assembly is identical to the knee module except in one aspect. The number of vibration units is different, there are at most 2 vibration units. The vibration units are arranged to maximize sensation in major muscle groups around the ankle joint. The present invention targets the extensor Capri group, flexor Capri group, palmaris longus, extensor digitorum, extensor indicis (proprius), extensor digiti minimi (proprius), flexor digitorum superficialis, flexor digitorum profundus, and other muscles in the area as well as cutaneous receptors. The entire module is very similar to the band module.

According to an embodiment herein, the trunk module is an extension of the band module. The core is the same, with differences in the structure of the band to which the electronics are attached. The trunk module resembles a wide belt. Two vibration units are present on either side of the trunk.

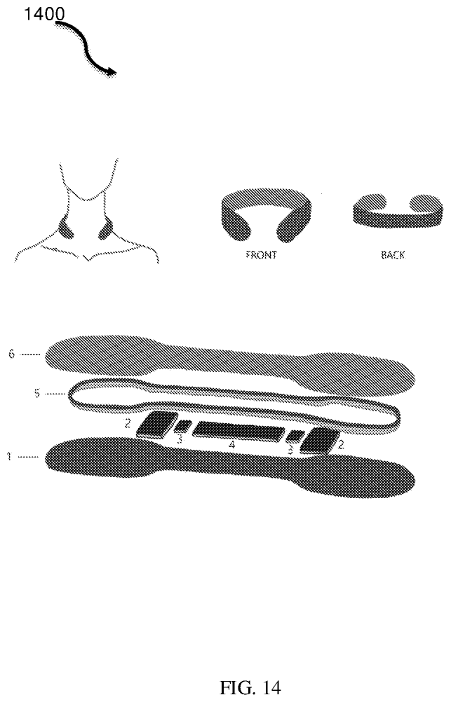

FIG. 14 illustrates a perspective view 1400 of a neck form factor, in accordance with at least one embodiment. Similar to the band module, the neck module, to be worn on the neck, has two vibration units. Further, the neck module has a stylistically different outer structure. Further, the neck module includes an outer mesh 1, various vibration units 2, various bone conduction units 3, battery housing unit and central control unit/power unit 4, bendable support structure 5, and an inner mesh 6.

The trunk and neck module have a monolithic form factor. The knee, ankle, sound, and wrist modules have a dual system form factor.

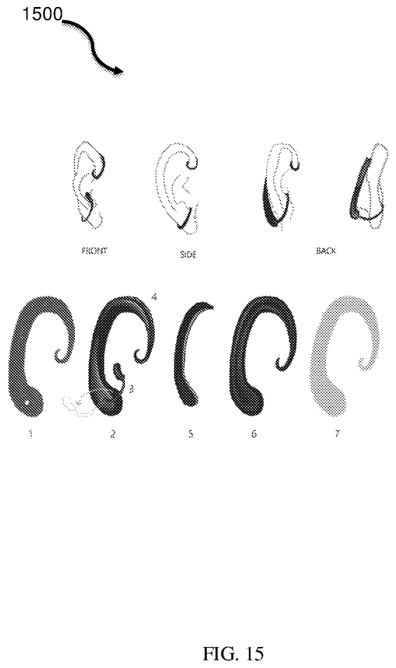

FIG. 15 illustrates a perspective view 1500 of an auditory device or sound module, in accordance with at least one embodiment. While the knee and other modules are compatible with off-the-shelf wireless and wired speakers/earphones/headphones, a separate sound module also exists. The sound module fits around the ear and has to cushion for a comfortable fit. It is compatible with eyeglasses and has a distinct shape to accommodate them. It uses bone and cartilage conduction techniques to transmit sound without reducing situational awareness. In an embodiment, the sound module includes one or more speaker units for providing emergency alerts. The structure of the sound module contains the central module PCB assembly along with a sound conduction transducer. Further, the sound module includes an outer cushioning 1, an outer shell 2, an adjustable claim 3, a groove for special stem 4, various bone conduction units, cartilage conduction unit, sound conduction unit, battery housing unit, and central control unit/power unit 5, an inner shell 6, and an inner cushioning 7. In an embodiment, the sound module includes an IMU and to capture gait data. In an embodiment, the sound module includes one or more EMG sensors and EEG sensors. and electrodes. In an embodiment, the sound module includes a locking arm mechanism for a good fit.

In an embodiment, the sound module has compatibility with spectacles and provides cushioning around the ear. Further, the sound module provides a mechanism for the change of position of the bone/cartilage conduction unit.

In an embodiment, the present system includes a smart band in which all electronics are arranged in a thick band. The smart band can go around the thigh, wrist, ankle. Multiple can link around the trunk. The central device with a single vibration unit can go into a necklace, other smaller form factors.

In an embodiment, the digital platform of the present invention addresses the needs of three stakeholders: patients, family (and caregivers), and healthcare professionals.

For patients, the digital platform provides SOS and alert service, enables fast communication with caregivers, manages psychological symptoms such as depressions, anxiety, and loneliness, and provides engaging activities to stem the progression of dementia.

For family and caregivers, the digital platform provides alerts and other useful information, provides insights about conditions, recommendations about clinic visits to reduce anxiety, and establishes a communication channel with healthcare professionals. Further, the digital platform provides tools to manage care such as arranging social interactions for patients, etc.

For healthcare professionals, the digital platform provides comprehensive data for symptoms, sleep patterns, falls, etc. beyond the clinic visit. The digital platform then provides insights into the progression, emergence of symptoms.

The digital platform is aimed towards reducing the burden of care and enhancing therapy through data. Parkinson's is a disease that affects the entire family, and the present system is attempting to solve this larger problem of caring for chronic disease.

As used herein, and unless the context dictates otherwise, the term “configured to” or “coupled to” is intended to include both direct coupling (in which two elements that are coupled to each other contact each other) and indirect coupling (in which at least one additional element is located between the two elements). Therefore, the terms “configured to”, “configured with”, “coupled to” and “coupled with” are used synonymously. Within the context of this document terms “configured to”, “coupled to” and “coupled with” are also used euphemistically to mean “communicatively coupled with” over a network, where two or more devices can exchange data with each other over the network, possibly via one or more intermediary device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Thus, the present gait moderation aiding system provides a digital platform that communicates wirelessly with the wearable device and smart stick and provides analytics to medical professionals as well as users. Further, the present gait moderation aiding system provides timed vibratory stimulus to knee tendons and coordinated auditory stimulus. Furthermore, the present gait moderation aiding system provides visual stimulus using the laser on the smart stick. Additionally, the present gait moderation aiding system provides various advantages such as adaptation to the user's gait through Machine Learning algorithm embedded on the wearable device, terrain mapping and freezing prediction on the smart stick, and modulated stimulus to help alleviate occurrences of freezing and reduce the time of freezing episodes.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms enclosed. On the contrary, the intention is to cover all modifications, alternative constructions, and equivalents falling within the spirit and scope of the invention, as defined in the appended claims. Thus, it is intended that the present invention cover the modifications and variations of this invention, provided they are within the scope of the appended claims and their equivalents.

1. A gait moderation aiding system (100) operable to support body posture of a user, said system characterized in that the gait moderation aiding system (100) comprises:
a smart stick (102) to provide a visual cue upon detection of a gait irregularity of the user, wherein the smart stick (102) comprises:
an elongated housing (222) comprising a proximal end and a distal end;
a handle (201) coupled to the proximal end of the elongated housing (222), wherein the handle (201) comprising an ergonomic grip (202), and a plurality of pressure sensors (312) placed on the ergonomic grip (202) to detect grip and grip strength of the user, wherein the handle (201) comprises a concealed push button (208) to generate vibration upon receiving a press gesture from the user, wherein the handle (201) comprises a first printed circuit board (PCB) (315);
a base (204) coupled to the distal end of the elongated housing (222), wherein the base (204) houses a second printed circuit board (PCB) (316), a gait detection sensor (318), and one or more terrain detection sensors (320), wherein the gait detection sensor (318) comprising an Inertial Measurement Unit (IMU) (322) to detect gait irregularity of the user by measuring orientation of the user's body, wherein the terrain detection sensors (320) comprising one or more infrared rangefinders (324) to detect terrain to provide the visual cue to guide the user while walking;
a shaft (210) connected with the handle (201) to house a wiring harness to connect the first and second printed circuit boards (PCBs) (315, and 316) to a plurality of primary peripherals (344); and
a central device (104) wirelessly connected with the smart stick (102) to assess the gait irregularity of the user to provide a vibratory cueing, and an auditory cueing, wherein the central device (104) is coupled to a wearable device (306) having a plurality of wearable form factors based on body parts of the user, wherein the central device (104) provides adaptive vibratory cueing, and auditory cueing through an auditory device (110), wherein the wearable device (306) logs sensory data to build a balance profile for the user, wherein the central device (104) comprises an electronic assembly (302) to house a processor (304) that is configured to:
control a plurality of secondary peripherals (108);
execute a plurality of instructions to process sensory data received from a plurality of detectors coupled to the wearable device (306) to assess the gait irregularity of the user, wherein the detectors are housed in the electronic assembly (302);
establish a communication with the smart stick (102);
train and run a plurality of machine learning models (404); and
actuate a plurality of transducers (310) comprising a vibration transducer, and a sound transducer; and trigger the vibratory cueing, and the auditory cueing;
a computing device (106) connected with the smart stick (102) and the central device (104) over a network, wherein the computing device (106) comprises a memory (120) and a processing unit (122) configured to execute a communication application to present gait analytics to a caregiver, and a medical professional; and
a control algorithm (302) to control the transducers (310), wherein the control algorithm (302) comprising a machine learning model (404) and a statistical model (406) to perform data analysis on the sensory data, wherein the machine learning model (404) is trained to perform supervised prediction of fall risk based on the sensory data, wherein the machine learning model (404) is trained and transmitted to the communication application that updates the machine learning model (404) in the processor (304), and the processing unit (122).

2. The gait moderation aiding system (100) as claimed in claim 1, wherein the communication application provides a digital platform to establish an interaction between the user, caregiver, and the medical professional.

3. The gait moderation aiding system (100) as claimed in claim 1, wherein the base (204) of the smart stick (102) comprises a microcontroller (326) to process data captured from the sensors to output stimuli.

4. The gait moderation aiding system (100) as claimed in claim 1, wherein the IMU (322) comprises one or more accelerometers, one or more gyroscopes, and one or more magnetometers.

5. The gait moderation aiding system (100) as claimed in claim 1, wherein the base (204) comprises a flex sensor, a plurality of infrared (IR) sensors, a plurality of ultrasonic sensors, and an assembly of pressure sensor to detect an angle of deflection or bending of a surface while walking.

6. The gait moderation aiding system (100) as claimed in claim 1, wherein the base (204) comprises a broad-spectrum camera (330) to capture a range of wavelengths and operate in infrared (IR) waves, ultraviolet (UV) waves, and visual spectrum.

7. The gait moderation aiding system (100) as claimed in claim 1, wherein the smart stick (102) comprises a plurality of momentum stabilizer wheels (338) to stabilize the smart stick (102) upon an occurrence of freezing of gait and detecting loss of balance.

8. The gait moderation aiding system (100) as claimed in claim 1, wherein the base (204) of the smart stick (102) comprises a plurality of time-of-flight radar sensors.

9. The gait moderation aiding system (100) as claimed in claim 1, wherein the base (204) of the smart stick (102) comprises a plurality of laser emitters (340) to emit a laser for the visual cueing.

10. The gait moderation aiding system (100) as claimed in claim 1, wherein the base (204) comprising a plurality of legs (230) to provide a vertical support to the smart stick (102).

11. The gait moderation aiding system (100) as claimed in claim 1, wherein the base (204) and the shaft (210) houses a battery assembly to power the first and second PCBs, the gait detection sensor, and the terrain detection sensors.

12. The gait moderation aiding system (100) as claimed in claim 1, wherein the central device (104) comprises a power source to power the electronic assembly (302).

13. The gait moderation aiding system (100) as claimed in claim 1, wherein the wearable device (306) comprises a fabric.

14. The gait moderation aiding system (100) as claimed in claim 1, wherein the electronic assembly (302) of the central device (104) is arranged over the wearable form factors that are designed to be worn on and around one or more of knee joint, ankle, wrist, neck, ear, and trunk.

15. The gait moderation aiding system (100) as claimed in claim 1, wherein the wearable form factors comprising: a knee form factor, an ankle form factor, a wrist form factor, a neck form factor, an ear form factor, and a trunk form factor.

16. The gait moderation aiding system (100) as claimed in claim 1, wherein the second PCB (316) is placed in a padded housing on the base (204).

17. The gait moderation aiding system (100) as claimed in claim 1, wherein the handle (201) comprises a plurality of mechanical buttons (314) to adjust a distance of the laser and control at least one of an audio signal and a user interface (UI) of the computing device (106).

18. The gait moderation aiding system (100) as claimed in claim 1, wherein the handle (101) comprising a touch sensor placed on the ergonomic grip to detect a touch of the user.

19. The gait moderation aiding system (100) as claimed in claim 1, wherein the plurality of primary peripherals (344) comprising a vibration unit.

20. The gait moderation aiding system (100) as claimed in claim 1, wherein the plurality of secondary peripherals (108) comprising a sound system and a haptic vibration system.
